(19) United States
(12) Patent Application Publication
(10) Pub. No.: US 2021/0046326 A1
(43) Pub. Date: Feb. 18, 2021

(54) PATIENT SUPPORT MECHANISM FOR PHOTONIC THERAPY

(71) Applicant: RICHARD OGDEN DEROBERTS, WARDENSVILLE, WV (US)

(72) Inventor: RICHARD OGDEN DEROBERTS, WARDENSVILLE, WV (US)

(21) Appl. No.: 16/543,385

(22) Filed: Aug. 16, 2019

(51) Int. Cl. A61N 5/06

(52) U.S. Cl. CPC A61N 5/062; A61N 2005/067; A61N 5/0617

(57) ABSTRACT

Method of producing a mechanism for supporting a patient while therapeutic laser or light emitting diode energy is administered providing a comfortable, relaxing position for the patient, cooling for the laser or light emitting diode emitter device and ensuring the optimum distance between the patient treatment area and the laser or light emitting diode emitter device so that correct pressure against the patient treatment area is achieved

### CROSS-REFERENCE TO RELATED APPLICATIONS

Ser. No. 13/690,706 U.S. Pat. No. 8,784,462 B2 Ser. No. 14/271,511 U.S. Pat. No. 9,126,034 B1

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

### 1. Technical Field of Invention

Embodiments in accordance with the invention relate generally to phototherapy. Phototherapy is a therapeutic physical modality using photons from the visible and infrared spectrum for tissue wound and burn healing, pain reduction, rhytide reduction (skin wrinkle) and hair follicle growth. It has also been shown to induce adipose cell membrane pore creation thereby allowing triglycerides, glycerol and free fatty acids to transit across the membrane into interstitial space. There have been more than 4000 studies published worldwide on the benefits of low level laser therapy (LLLT) and the effects observed with therapeutic lasers. Photo-bio-modulation increases ATP synthesis by changing the oxidation/reduction status of the mitochondria and activates the sodium/potassium pump thereby altering cell membrane permeability to calcium. Cell growth has been stimulated by an increase in cell metabolism. Higher levels of cell regeneration have been documented. LLLT has been shown to stimulate nerve function and the production of nitric oxide and endorphins. The neuropeptide substance P (SP) and histamine have been shown to be reduced thereby reducing local inflammatory response. LLLT also reduces the formation of acetylcholine, and bradikynin. LLLT has also been shown to reduce fibrous tissue formation. In photodynamic therapy (PDT) a photosensitizer is mixed with antibodies that are targeted to antigens on abnormal tissue. This mixture is then administered to the patient and binds with the antigens. Radio magnetic radiation having a wavelength corresponding to the absorption wavelength of the photosensitizer is then administered to the patient. This treatment reduces the size of the abnormal tissue.

### 2. Description of Related Art

Low level laser therapeutic instruments (LLLTI) achieve their therapeutic effect by emitting laser or light emitting diode (LED) radiation at a chosen frequency or frequencies at a chosen power level for a chosen period of time at a chosen distance over a chosen area. Generally laser or LED power is measured in watts, area is measured in centimeters (cm) squared, distance is measured in centimeters and time is measured in seconds. Therapeutic dosage is measured in watts multiplied by seconds divided by area in cm squared. Watts multiplied by seconds is defined as joules so dosage then is joules/cm². From this we see that to apply a larger dosage to the same area we can either increase the power of the laser or LED or the length of time the laser or LED light is applied, or both.

All semiconductor lasers and LEDs produce heat when energized. Edge emitting lasers produce more heat than VCSEL or HCSEL devices or LEDs because they are less efficient. Heat causes lasers to reduce their laser power output and to shift their laser light frequency to longer wavelengths. It has been shown that by changing the frequency only slightly the effect of the laser light can be reduced by 80% or more. This reduction of the light affect combined with the reduction of power output of the laser caused by rising temperature can render the LLLTI completely ineffective. Automatic power control systems which monitor the laser power output and try to maintain a constant power output exacerbate the problem by increasing the electrical energy supplied to the laser diode thereby further increasing the heat generated. Without cooling, laser diodes can ‘run away’ and burn out immediately or have their life span reduced dramatically. The temperature of a patient's treatment area can vary significantly. The temperature of the hand or foot can be 20 degrees Fahrenheit cooler than the chest. Also room temperature will cause differences in skin temperature. Different body physiques will cause significant differences in skin temperature from one patient to the next. The only way to ensure that the laser diodes or LEDs are radiating at the optimum frequency is with active temperature control. Small hand held LLLTI require more time for the treatment of a given area because they must be moved repeatedly. Because most large LLLTI are not flexible they do not apply an even and precise dosage to any part of the treatment area which is curved or contains small hollows. In the case of both small LLLTI and most large area LLLTI the instruments are held in place by either the patient's or technician's hand or laid onto the patient in a horizontal manner and kept in place by gravity. In the case of scanning LLLTI the laser beam is spread over a large area and requires a high power laser applied for a long period of time to administer the same dosage. Scanning LLLTI do not apply an even and precise dosage pattern because the laser diode is not a constant distance from the entire treatment area and because a scan line contains more laser energy in the center of the scan line than at either of the ends of the scan line.

Several newer LLLTI designs have the ability to conform to the contours of a patient's body but are problematic for several reasons. These LLLTI position the lasers in contact with the patient's skin or very close to the skin. This positioning concentrates the laser beam in a small diameter at the center of the treatment area because the beam does not have room to expand over the entire treatment area. Some designs employ vertical cavity surface emitting lasers (VCSEL) or horizontal cavity surface emitting lasers (HCSEL) devices. These lasers project a very narrow beam with almost no beam divergence and cannot spread their light energy over the entire treatment area without optical lenses which these LLLTI do not employ. In addition these designs are made of non-breathable materials held in direct contact with the skin. In some cases these LLLTI are intended to be worn for many hours at a time and in some cases days at a time. This can cause skin rashes, be extremely uncomfortable, retard blood flow in the area, and cause sweating which can attenuate the laser light. Some of these LLLTI are programmed to energize at specific time intervals during the day and night. If the LLLTI has been removed by the patient in order to bathe or because of discomfort the LLLTI will not recognize this and run its programmed course of treatment without the patient being involved.

### BRIEF SUMMARY OF THE INVENTION

A typical embodiment in accordance with the invention provides a mechanism for supporting a patient while therapeutic laser or light emitting diode energy is administered. The support mechanism (SM) includes a soft, flexible air entry frame (1) or (14) and a soft, flexible air exit frame (3). Between these two frames is inserted the laser or light emitting diode emitter device (7). A solid backing board (6) is attached to the back of the air entry frame (1) or air entry frame (14). This combination of four components comprises the SM (9). If the laser or light emitting diode emitter device (7) employs fans (19) for forcing cooling air through entry air tubes (2), the light emitting devices (8) and out exit air tubes (20) the air entry frame (1) will be employed. If the laser or light emitting diode emitter device (7) does not employ fans for forcing cooling air through the light emitting devices (8) then air entry frame (14) is employed in conjunction with air attachment head (17), air duct tube (15) and air compressor (16) to effect cooling. Air entry frame (1) has a cavity (4) to allow the laser or light emitting diode emitter device (7) to recede back into the air entry frame to allow correct pressure against the patient treatment area. The air exit frame (3) has a cavity (5) with positioning notches (13) to hold the light emitting diode emitter device (7) in correct alignment and facilitate light transmission onto the patient treatment area. The SM (9) allows a patient to sit back against the laser or light emitting diode emitter device (7) or lye upon the laser or light emitting diode emitter device (7) in relaxed comfort while keeping the laser or light emitting diode emitter device (7) the correct distance from the patient treatment area and cooling the light emitting devices (8) ensuring optimum temperature for light emission. Patient table (10) in conjunction with multiple soft, flexible pads (12) and the SM (9) allow a patient to lye comfortably upon the laser or light emitting diode emitter device (7). This scenario allows a patient to treat a shoulder, upper back, lower back, hip, thigh or calf. A standard household chair (18) in conjunction with one soft, flexible pad (12) and the SM (9) allows a patient to lean back comfortably against the SM (9) for lower or upper back treatment. The SM (9) can be incorporated into the design and manufacture of a reclining chair (11), work chair (11), sofa chair (11) or hospital bed (11) in conjunction with the air attachment head (17), air duct tube (15) and air compressor (16).

### BRIEF DESCRIPTION OF THE SEVERAL VIEWS OF THE DRAWINGS

FIG. 1 is a view showing one possible embodiment of the air entry frame for use with laser or light emitting diode emitter devices employing fans for cooling.

FIG. 2 is a view showing one possible embodiment of the air exit frame.

FIG. 3 is a view showing one possible embodiment of the air entry frame in conjunction with the air attachment head, air duct tube and air compressor.

FIG. 4 is a view showing one possible embodiment of the solid backing board.

FIG. 5 is a view showing one possible embodiment of a laser or light emitting diode emitter device, light emitting devices and the one or more fans.

FIG. 6 is a view showing one possible embodiment of the solid backing board, air entry frame, laser or light emitting diode emitter device and air exit frame as separate entities and as an assembled SM.

FIG. 7 is a view showing one possible embodiment of an assembled SM from the light emitting side. This side faces the patient treatment area.

FIG. 8 is a view showing one possible embodiment of an assembled SM from the back side. This side rests against a chair back or table top.

FIG. 9 is a view showing one possible embodiment showing a soft, flexible pad.

FIG. 10 is a view showing one possible embodiment showing a patient table.

FIG. 11 is a view showing one possible embodiment showing a standard household chair.

FIG. 12 is a view showing one possible embodiment showing a reclining chair, work chair, sofa chair or hospital bed incorporating the SM.

FIG. 13 is a view showing one possible embodiment of the present invention with all the major assemblies depicted in a usable arrangement as a bench or table.

FIG. 14 is a view showing one possible embodiment of the present invention with all the major assemblies depicted in a usable arrangement as a chair.

FIG. 15 is a view showing one possible embodiment of the present invention with all the major assemblies depicted in a usable arrangement as a reclining chair, work chair, sofa chair or hospital bed.

### DETAILED DESCRIPTION OF THE INVENTION

The following references to the drawings are discussed in the narrative below.
1 Air entry frame for use with laser or light emitting diode emitter devices employing fans for cooling
2 Air entry tubes
3 Air exit frame
4 Air entry frame cavity
5 Air exit frame cavity
6 Solid backing board
7 Laser or light emitting diode emitter device
8 Light emitting devices
9 Support mechanism (SM)
10 Patient table
11 Reclining chair, work chair, sofa chair or hospital bed
12 Soft, flexible pad
13 Exit frame cavity positioning notches
14 Air entry frame for use with laser or light emitting diode emitter device not employing fans for cooling
15 Air duct tube
16 Air compressor
17 Air attachment head
18 Standard household chair
19 Fan
20 Air exit tubes

The present disclosure relates to producing a mechanism for supporting a patient while therapeutic laser or light emitting diode energy is administered. Specific examples of tubing, padding, materials, and other arrangements are described below to simplify the present disclosure. These are, of course, merely examples and are not intended to limit the invention from that described in the claims.

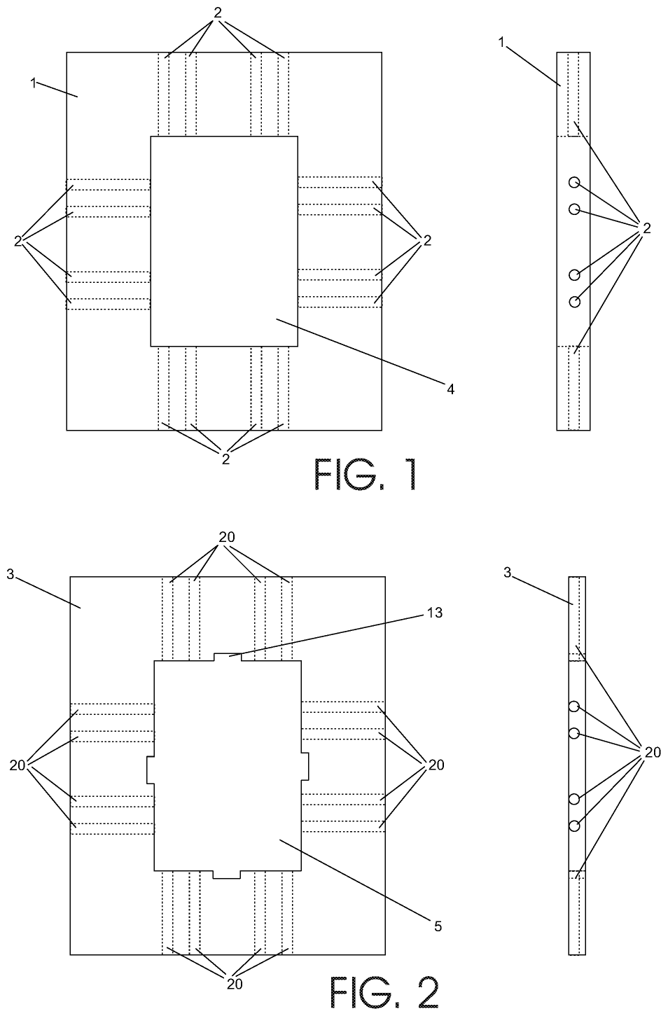

Referring now to FIG. 1 and FIG. 5 of the drawings, the reference numeral (1) refers to a flexible closed cell material comprising an air entry frame. The term flexible in this context means the ability to bend in an arc with radius of 4 inches or less without permanently deforming. The term closed cell membrane in this context means a material similar to polyethylene foam, neoprene foam, polystyrene foam and others. The air entry frame (1) has air entry tubes (2) molded into it, which draw air in from the surrounding area in order to promote cooling. The air entry frame (1) also has a cavity (4) molded into it to allow the laser or light emitting diode emitter device (7) to recede back into the air entry frame (1) so that correct pressure against the patient treatment area is achieved.

Referring now to FIG. 2 and FIG. 5 of the drawings, the reference numeral (3) refers to a flexible closed cell material the same as the air entry frame (1) comprising an air exit frame (3). The air exit frame (3) has air exit tubes (20) molded into it, which channel air to the surrounding area in order to promote cooling. The air exit frame (3) also has a cavity (5) molded into it to allow the laser or light emitting diode emitter device (7) to emit photonic therapy light onto the patient treatment area. The cavity (5) has positioning notches (13) to position and align the laser or light emitting diode emitter device (7).

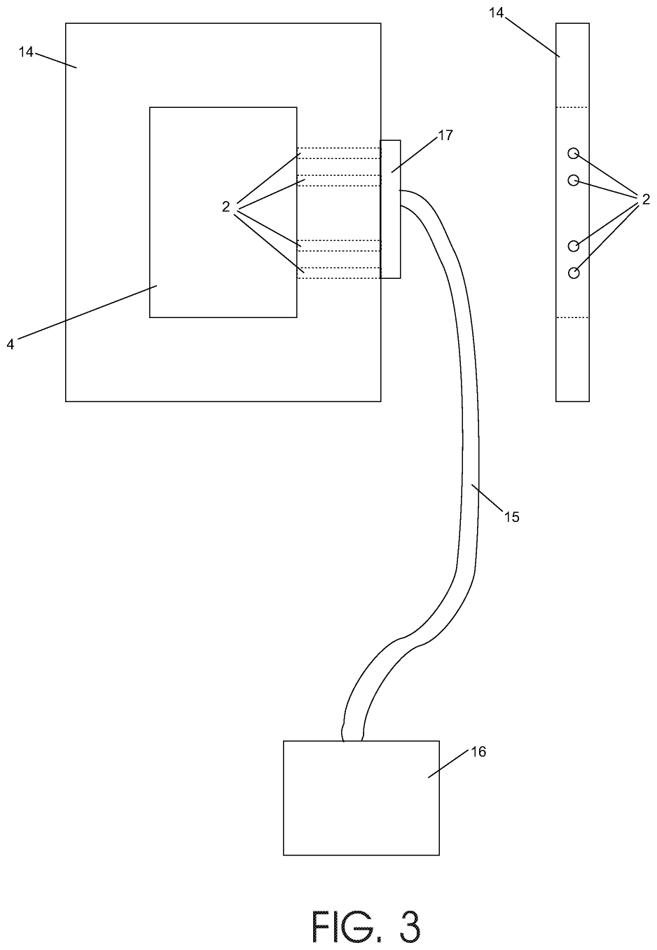

Referring now to FIG. 3 and FIG. 5 of the drawings, reference numeral (14) refers to a flexible closed cell material the same as air entry frame (1) comprising an air entry frame (14) that is used in conjunction with a laser or light emitting diode emitter device (7) not employing fans for cooling. Air entry tubes (2) are connected to an air attachment head (17) which is connected to an air duct tube (15) which is connected to an air compressor (16) which provides the cooling air for the laser or light emitting diode emitter device (7). The air entry frame (14) also has a cavity (4) molded into it to allow the laser or light emitting diode emitter device (7) to recede back into the air entry frame (14) so that correct pressure against the patient treatment area is achieved.

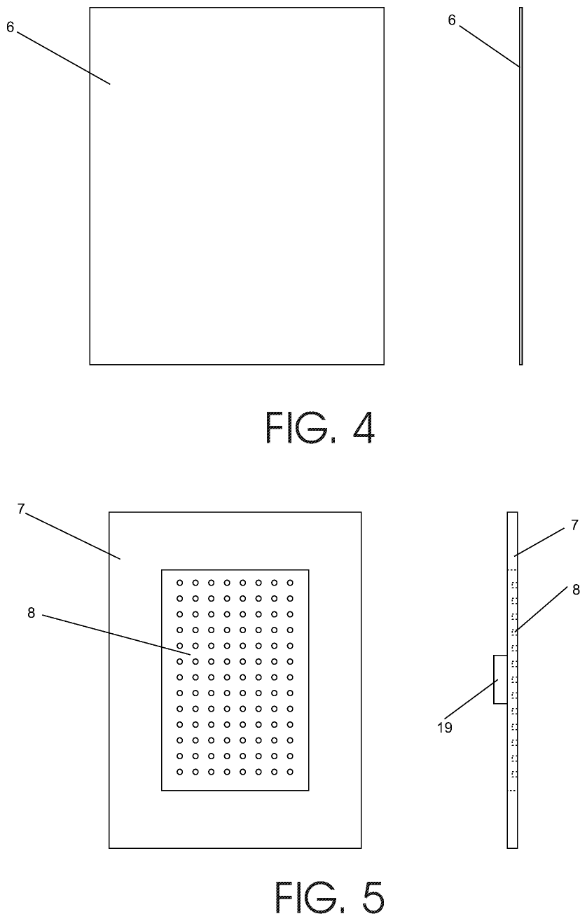

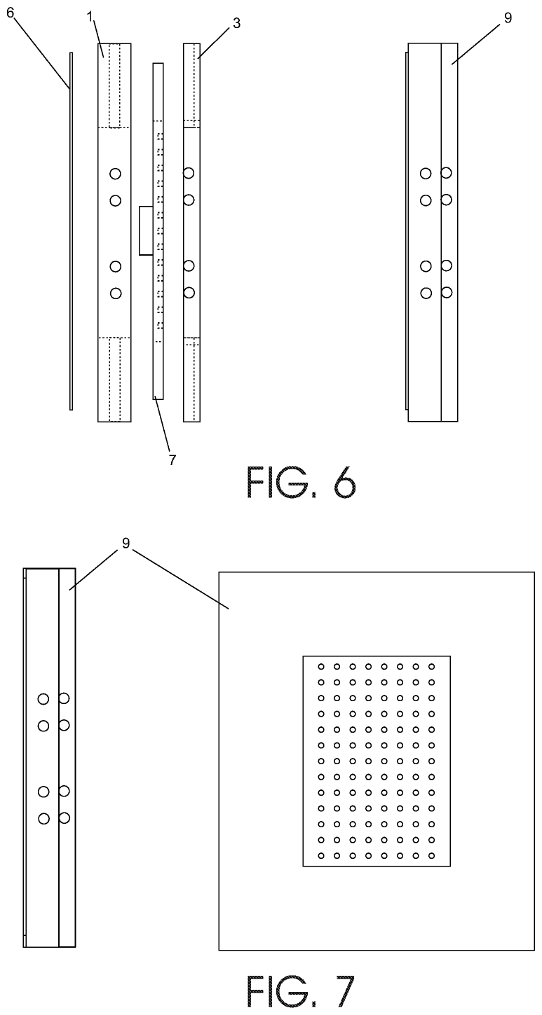

Referring now to FIG. 4, FIG. 5 and FIG. 6 of the drawings, the reference numeral (6) refers to a solid backing board (6). The solid backing board (6) keeps the cooling air from escaping through the back of the SM (9), maintains a clearance area between the object on which the SM (9) rests and the cooling fans (19) and provides structural rigidity to the entire SM (9). The solid backing board can be made of thin plywood, MDF, or pressed particle board, etc.

Referring now to FIG. 5 of the drawings, the reference numeral (7) refers to a laser or light emitting diode emitter device (7). This device is referenced as the source of the photonic therapy light and can be any light source which can be fitted to the SM (9). Reference numeral (8) refers to any light producing diode and reference numeral (19) refers to one or more cooling fans which may or may not be utilized by the laser or light emitting diode emitter device (7).

Referring now to FIG. 6 of the drawings, the reference numeral (6) refers to the solid backing board, reference numeral (1) refers to the air entry frame for use with laser or light emitting diode emitter devices employing fans for cooling, reference numeral (7) refers to the laser or light emitting diode emitter device and reference numeral (3) refers to the air exit frame. This FIG. displays the order of their assembly into a completed SM (9).

Referring now to FIG. 7 of the drawings, the reference numeral (9) refers to a completed SM (9) showing a side view and a view of the SM (9) side facing the patient treatment area.

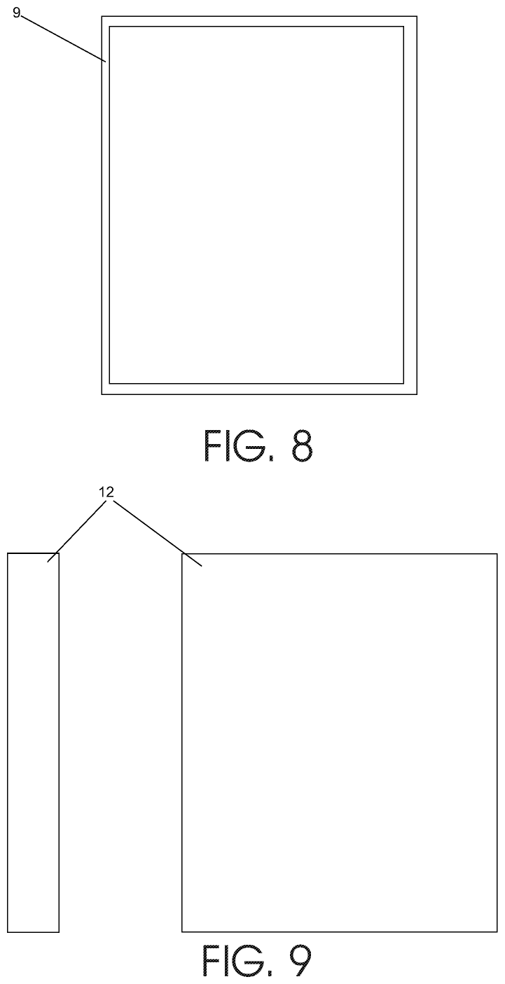

Referring now to FIG. 8 of the drawings, the reference numeral (9) refers to the side of the SM (9) facing away from the patient treatment area. This side rests on a supporting object such as a chair or table.

Referring now to FIG. 9 and FIG. 6 of the drawings, the reference numeral (12) refers to one of the multiple soft, flexible pads. These pads are made of closed cell material the same as the air entry frame (1), air entry frame (14) and the air exit frame (3).

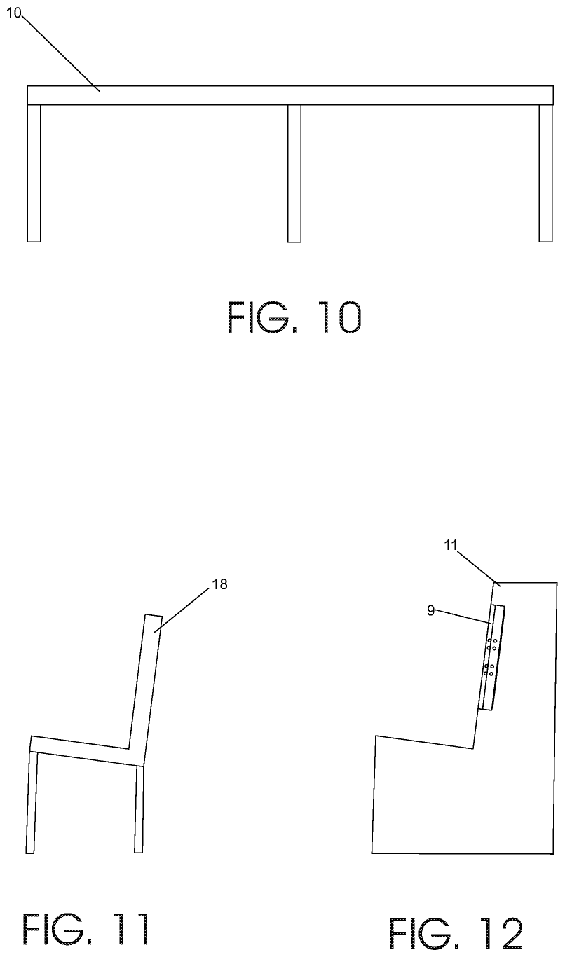

Referring now to FIG. 10 of the drawings, the reference numeral (10) refers to a patient table. This device is shown as a supporting device for the intended use of the SM (9) and the flexible pads (12).

Referring now to FIG. 11 of the drawings, the reference numeral (18) refers to a standard household chair. This device is shown as a supporting device for the intended use of the SM (9).

Referring now to FIG. 12 of the drawings, the reference numeral (11) refers to the reclining chair, work chair, sofa chair or hospital bed. This device is shown as a supporting device for the intended use of the SM (9). Reference numeral (9) refers to the SM (9) as an integral part of the reclining chair, work chair, sofa chair or hospital bed as designed by the manufacturer and assembled as a complete unit. That is, the manufacturer designed the reclining chair, work chair, sofa chair or hospital bed to incorporate and accommodate the SM (9).

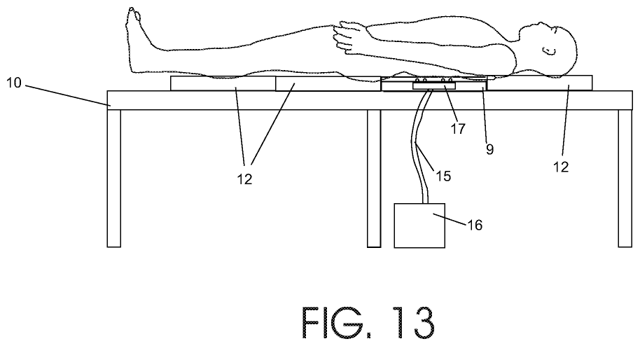

Referring now to FIG. 13 of the drawings, the patient table (10), multiple soft, flexible pads (12), SM (9), air attachment head (17), air duct tube (15) and air compressor (16) are shown as a complete working configuration as intended to be used for a patient lying upon the SM (9).

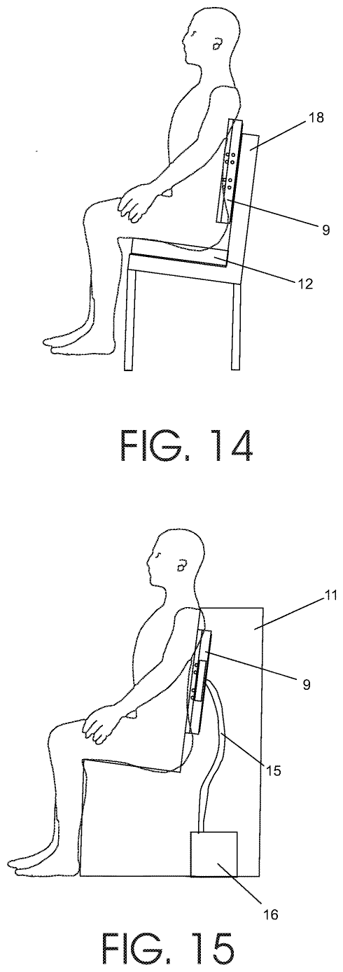

Referring now to FIG. 14 of the drawings, a standard household chair (18), one soft, flexible pad (12) and the SM (9) are shown as a complete working configuration as intended to be used for a patient leaning back against the SM (9).

Referring now to FIG. 15 of the drawings, the reclining chair, work chair, sofa chair or hospital bed (11), SM (9), air attachment head (17), air duct tube (15) and air compressor (16) are shown as a complete working configuration as intended to be used for a patient reclining upon the SM (9).

What is claimed is:

1. A mechanism for supporting a patient (support mechanism) while therapeutic laser or light emitting diode energy is administered providing a comfortable, relaxing position for the patient, cooling for the laser or light emitting diode emitter device and ensuring the optimum distance between the patient treatment area and the laser or light emitting diode emitter device so that correct pressure against the patient treatment area is achieved, consisting of a soft, flexible air entry frame with air entry tubes for circulating cooling air through the laser or light emitting diode emitter device, in which is molded a hollow cavity to provide space for the one or more cooling fans;
a second soft, flexible air exit frame with air exit tubes for circulating cooling air through the laser or light emitting diode emitter device, in which is molded a hollow cavity to allow the laser or light emitting diode emitter device to emit photonic therapy light onto the patient treatment area, the cavity has positioning notches to position and align the laser or light emitting diode emitter device;
a solid backing board to keep the cooling air from escaping from the back of the support mechanism, maintain a clearance area between the object on which the support mechanism rests and the cooling fans and provide structural rigidity to the entire support mechanism.

2. The support mechanism of claim 1 wherein the flexible air entry frame is replaced with a soft, flexible air entry frame with air entry tubes for circulating cooling air through the laser or light emitting diode emitter device, in which is molded a hollow cavity to provide space for air to circulate through the laser or light emitting diode emitter device, connected to an air attachment head which is connected to an air duct tube which is connected to an air compressor which causes cooling air to be supplied to the laser or light emitting diode emitter device.

3. Soft, flexible pads to support the patient's body in conjunction with the support mechanism to provide entire body support.

4. The support mechanism of claim 2 wherein the support mechanism is imbedded into the structure of a reclining chair, work chair, sofa chair or hospital bed specifically designed for therapeutic laser or light emitting diode energy administration.

5. A reclining chair, work chair, sofa chair or hospital bed specifically designed for therapeutic laser or light emitting diode energy administration.
